(19) United States
(12) Patent Application Publication
(10) Pub. No.: US 2018/0360713 A1
(43) Pub. Date: Dec. 20, 2018

(54) COMPOSITION COMPRISING AT LEAST TWO FATTY ACID ESTERS OF (POLY)GLYCEROL, AND USE THEREOF IN COSMETICS

(71) Applicant: L'OREAL, Paris (FR)

(72) Inventors: Chantal JOUY, Chevilly La Rue (FR); Christian TRAN, Aulnay-sous-Bois (FR)

(73) Assignee: L'OREAL, Paris (FR)

(21) Appl. No.: 16/061,849

(22) PCT Filed: Dec. 16, 2016
(86) PCT No.: PCT/EP2016/081623

§ 371 Date: Jun. 13, 2018

(30) Foreign Application Priority Data
Dec. 18, 2015 (FR) 1562744

(51) Int. Cl. A61K 8/37; A61K 8/34; A61K 8/46; A61K 8/39; A61K 8/92; A61K 8/06; A61Q 19/08

(52) U.S. Cl. CPC A61K 8/375; A61K 8/345; A61K 8/466; A61K 8/39; A61K 2800/5922; A61K 8/068; A61Q 19/08; A61K 8/062; A61K 2800/21; A61K 8/922

(57) ABSTRACT

The invention relates to a composition in the form of a nanoemulsion or micro-emulsion, comprising: at least two different fatty acid esters of (poly)glycerol a) and b); c) at least one polyol in a total content of greater than 15% by weight; d) at least one oil; e) water; and f) at least one anionic surfactant of formula (II) it being understood that said composition comprises at least one anionic surfactant of formula (II) when the first fatty acid ester of (poly)glycerol is a polyglyceryl laurate comprising from 3 to 6 glycerol units and the second fatty acid ester of (poly)glycerol is a (poly)glyceryl laurate comprising from 1 to 3 glycerol units. The invention also relates to a cosmetic treatment process for reducing and/or preventing the signs of ageing of keratin materials such as the skin, by application of such a composition.

The subject of the present invention is a composition, in particular a cosmetic composition, in the form of a nanoemulsion or of a microemulsion, comprising at least two different fatty acid esters of (poly)glycerol, at least 15% of polyol(s), at least one oil, water, and optionally at least one anionic surfactant of formula (II), and the use of this composition for treating and/or preventing the signs of skin ageing.

Women and men currently have a tendency to wish to appear youthful for as long as possible and consequently seek to tone down the signs of ageing on the skin, which are reflected in particular by wrinkles and fine lines. In this regard, the advertising and fashion industries mention products for retaining radiant and wrinkle-free skin, signs of youthful skin, for as long as possible, all the more so since physical appearance has an effect on the psyche and/or on morale.

Hitherto, wrinkles and fine lines were treated using cosmetic products containing active agents acting on the skin, for example by improving its cell renewal or alternatively by promoting the synthesis, or preventing the degradation, of the elastic fibres which make up skin tissue.

The skin is constituted of two compartments, a surface compartment, the epidermis, and the other deeper compartment, the dermis, which interact. Natural human epidermis is composed mainly of three types of cells, namely keratinocytes, which form the vast majority, melanocytes and Langerhans cells. Each of these types of cells contributes, by virtue of its intrinsic functions, to the essential role played in the body by the skin, in particular the role of protecting the body against external attacking factors, which is known as the “barrier function”.

The epidermis is conventionally divided into a basal layer of keratinocytes that constitutes the germinative layer of the epidermis, a spinous layer constituted of several layers of polyhedral cells positioned on the germinative layers, one to three “granular” layers constituted of flattened cells containing distinct cytoplasmic inclusions, keratohyalin granules, and finally the cornified layer (or stratum corneum), constituted of a set of layers of keratinocytes at the terminal stage of their differentiation, known as corneocytes. Corneocytes are anuclear cells mainly constituted of a fibrous material containing cytokeratins, surrounded by a cornified envelope.

The dermis provides the epidermis with a solid support. It is also its nourishing element. It is constituted mainly of fibroblasts and of an extracellular matrix predominantly composed of collagen, elastin and a substance, known as ground substance, comprising glycosaminoglycans that are sulfated (e.g. chondroitin sulfate) or not (e.g. hyaluronic acid), proteoglycans and various proteases. These components are synthesized by the fibroblasts. Leukocytes, mast cells or else tissue macrophages are also found therein. Finally, blood vessels and nerve fibres pass through the dermis. The cohesion between the epidermis and the dermis is provided by the dermo-epidermal junction.

The epidermis is constantly engaged in producing new keratinocytes to compensate for the continuous loss of epidermal cells at the cornified layer. However, in the course of ageing, a decrease in the number of cells in the proliferation phase, and consequently a decrease of the live epidermal layers, may be observed physiologically.

The homeostasis of the skin, and in particular of the epidermis, results from a finely regulated balance between the processes of proliferation and of differentiation of the skin cells. These processes of proliferation and differentiation are entirely regulated: they participate in the renewal and/or regeneration of the skin and lead to the maintenance of a constant thickness of the skin, and in particular of a constant thickness of the epidermis. This homeostasis of the skin also participates in maintaining the mechanical properties of the skin.

However, this homeostasis of the skin may be impaired by certain physiological factors (age, menopause, hormones, etc.) or environmental factors (UV stress, oxidative stress, irritant stress, etc.).

The proliferative cells are metabolically very active and are sensitive to these deleterious factors (intrinsic or environmental), with, as a consequence on the epidermis, a reduction in their amount. Certain biochemical markers characterise this loss of regenerative capacity of the epidermis, such as SAb-galactosidase activity (Dimri G P, et al. Proc Natl Acad Sci USA. 1995) or cell cycle impairment markers such as p16(INK4a) (Cordisco S & al J Invest Dermatol. 2010).

It is thus important to preserve this pool of cells in order to contribute toward delaying the onset of the signs of ageing.

The cell vitality of keratinocytes may be reduced in the context of ageing or because of oxidative stress (for example solar, i.e. UV, radiation, radiation in the visible range, infrared radiation), because of the epidermis being attacked by toxins or metabolites of the microflora, or, more generally, during chronological ageing. The capacity for renewal and differentiation of the keratinocytes is reduced and the homeostasis of structures dependent thereon, such as the barrier function of the epidermis, is impaired.

When the regenerative potential of the epidermis becomes smaller: the cells of the basal layer divide less actively, leading in particular to a slowing-down and/or decrease in epidermal renewal. Consequently, the cell renewal no longer compensates for the loss of cells removed at the surface, leading to atrophy of the epidermis and/or a reduction in skin thickness. This is likewise the case for the proliferative cells of the epidermal appendages, for example the nails, the consequence of which is a slowing-down of the growth of the nails.

With age, a barrier-function impairment develops, and a decrease in the expression of proteins playing a key role in the maintenance of this essential function of the skin, such as filaggrin, is observed.

Filaggrin is a protein resulting from the fragmentation of a molecule, profilaggrin, contained in the keratinocytes of the granular layer. Filaggrin expression is associated with the differentiation of epidermal cells, such as keratinocytes.

It allows the aggregation of keratin filaments and participates in the formation of the cell envelope of the cornified layer, and thus participates in the function of the skin barrier.

Impairments in epidermal homeostasis are also reflected by a dull and/or off-colour appearance of the skin complexion.

Impairment of the barrier function is manifested by various signs depending on the localization: dry skin, hyperkeratosis, thin epidermis, thin lips, surface wrinkles.

The disorders associated with impairment of the cellular vitality of the epidermis thus concern not only its structure, but also its homeostasis. The resistance to stress of the epidermis and its capacity for regeneration are reduced. If the skin barrier of an elderly person is compared with that of a young adult, the differences do not appear at first sight: the thickness of the cornified layer and the composition of its lipids are not necessarily altered, and the barrier function expressed by the transepidermal water loss is conserved. The deficiencies of the elderly skin barrier appear under mechanical stress or during exposure to irritant factors: the barrier of an elderly epidermis degrades more rapidly and its function recovers less quickly. On a daily basis, alcoholic disinfection, contact with lemon juice or other irritants then cause stinging and burning, and dry air is poorly tolerated, whereas a young skin tolerates this without any problem. An impaired skin barrier also facilitates the contact of allergens with the immune system of the epidermis, thus increasing the risk of allergic sensitization.

At the present time, there is sufficient evidence to prove that senescent cells accumulate with age in the body. Senescence-associated β-galactosidase is a marker of senescent cells and its accumulation has been shown in vivo in the skin (Dimai G P et al Proc Nat Acad. Sci. USA 1995; 92(20): 9363-7).

Another marker of senescence is the impairment of mitochondrial functioning. The role of the mitochondrion is to produce cellular energy.

The clinical signs of the phenomenon of photoageing have been widely described (Photodermatol Photoimmunol Photomed. 2008 (4) Fourtanier A., Moyal D., Seité S.).

Intrinsic ageing, also known as chronological ageing, of the skin is described as a result of an impairment in cellular vitality similar to that which takes place in the other organs. Intrinsic or chronological ageing is manifested by other clinical markers and signs, in particular impairment of the barrier function as described above (Farage M. A. et al. 2009; 10(2): 73-86).

These esthetic signs of skin ageing, such as dry skin, wrinkles, fine lines, etc., are such that there is a need in cosmetics for compounds acting on the skin to improve the cellular vitality when it is impaired.

Unexpectedly, it has been found, in the context of the present invention, that the particular compositions according to the invention described below are capable of stimulating filaggrin expression by normal human keratinocytes, and in particular of stimulating an increase in the thickness of the epidermis. These compositions will thus be particularly useful for preventing or limiting the consequences of skin ageing, including in particular the stimulation of keratinocyte proliferation and of the synthesis of the structural molecules of the skin, such as filaggrin.

The inventors have in particular discovered, unexpectedly, a composition in the form of a nanoemulsion or of a microemulsion, comprising at least two different fatty acid esters of (poly)glycerol, at least 15% of polyol(s), at least one oil, water, and optionally at least one anionic surfactant of formula (II), which is stable and has in particular good sensory properties, and which has in particular a transparent or slightly translucent appearance.

Consequently, the present invention relates to a composition, preferably a cosmetic composition, in the form of a nanoemulsion or microemulsion, comprising:

a) a first fatty acid ester of polyglycerol which is chosen from a fatty acid ester of polyglycerol formed from at least one acid comprising an alkyl or alkenyl chain containing from 12 to 20 carbon atoms and from 3 to 6 glycerol units;

b) a second fatty acid ester of (poly)glycerol which is chosen from a fatty acid ester of (poly)glycerol formed from at least one acid comprising an alkyl or alkenyl chain containing from 6 to 18 carbon atoms and from 1 to 3 glycerol units;

c) at least one polyol in a total content of greater than or equal to 15% by weight relative to the total weight of the composition;

d) at least one oil;

e) water; and

f) optionally at least one anionic surfactant of formula (II)

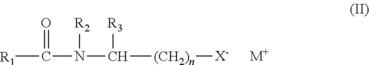

in which:

R₁ is a saturated or unsaturated, linear or branched alkyl chain having from 7 to 17 carbon atoms,

R₂ is H or a methyl group,

R₃ is H, COO⁻M⁺, MCH₂COO— or COOH,

n is 0 to 2,

X represents COO or SO₃⁻ and

M represents independently H, sodium, potassium or sorbitan;

it being understood that said composition comprises at least one anionic surfactant of formula (II) (f), preferably sodium N-methylstearoyltaurate, when the first fatty acid ester of polyglycerol (a) is a polyglyceryl laurate comprising from 3 to 6 glycerol units and the second fatty acid ester of (poly)glycerol (b) is a (poly)glyceryl laurate comprising from 1 to 3 glycerol units.

In one particular embodiment of the invention, the present invention relates to a composition as defined above, not comprising hesperetin or being free of hesperetin.

In one particular embodiment of the invention, the present invention relates to a composition as defined above, not comprising ceramids or being free of ceramids compounds.

Given that the cosmetic composition according to the present invention may have a transparent or slightly translucent appearance, the composition may preferably be used for lotions and the like. Furthermore, the cosmetic composition according to the present invention may produce a pleasant texture and afford moisturizing properties and also increased suppleness.

The composition, preferably the cosmetic composition, according to the present invention is described in greater detail hereinbelow.

The composition of the invention may be a cosmetic composition (i.e. intended for cosmetic purposes) or a dermatological composition. Preferentially, according to the invention, the composition is a cosmetic composition and even more preferentially a cosmetic composition for topical application.

The term “cosmetic composition” is in particular intended to mean a substance or a preparation intended to be brought into contact with the various superficial parts of the human body, in particular the epidermis, the bodily-hair and head-hair systems, the nails, the lips and the oral mucous membranes, with a view, exclusively or mainly, to cleansing them, making them more attractive, fragrancing them, modifying their appearance, protecting them, keeping them in good condition, or correcting body odours.

A subject of the present invention is also the cosmetic use of a composition as defined above, for preventing and/or reducing the signs of skin ageing, in particular the signs on the skin chosen from wrinkled skin, skin exhibiting impairment of its viscoelastic or biomechanical properties, skin exhibiting impairment in the cohesion of its tissues, thinned skin, and skin exhibiting impairment of its surface appearance.

Another subject of the present invention is a cosmetic treatment process for reducing and/or preventing the signs of ageing of keratin materials such as the skin, characterized in that a composition as defined above is applied to the keratin materials, preferably the skin, in particular mature and/or wrinkled skin.

Fatty Acid Esters of (Poly)Glycerol (a) and (b)

The composition according to the present invention comprises at least two fatty acid esters of (poly)glycerol that are different from one another.

The composition according to the invention comprises;

a) a first fatty acid ester of polyglycerol which is chosen from a fatty acid ester of polyglycerol formed from at least one acid comprising an alkyl or alkenyl chain containing from 12 to 20 carbon atoms and from 3 to 6 glycerol units, preferably from 5 to 6 glycerol units, and

b) a second fatty acid ester of (poly)glycerol which is chosen from a fatty acid ester of (poly)glycerol formed from at least one acid comprising an alkyl or alkenyl chain containing from 6 to 18 carbon atoms and from 1 to 3 glycerol units, preferably from 2 to 3 glycerol units.

The fatty acid esters of glycerol or polyglycerol used in the context of the present invention are non-ionic surfactants that are solid at a temperature of less than or equal to 45° C.

The compositions according to the invention comprise at least two fatty acid esters of glycerol or polyglycerol, which is optionally polyoxyalkylenated.

In the context of the present invention, mention may also be made of oxyalkylenated glycerol esters and in particular polyoxyethylenated derivatives of glyceryl esters of fatty acids and hydrogenated derivatives thereof. These oxyalkylenated glycerol esters can be chosen, for example, from glyceryl esters of fatty acids which are hydrogenated and oxyethylenated, such as PEG-200 hydrogenated glyceryl palmate, sold under the name Rewoderm LI-S 80 by the company Goldschmidt; oxyethylenated glyceryl cocoates, such as PEG-7 glyceryl cocoate, sold under the name Tegosoft GC by the company Goldschmidt, and PEG-30 glyceryl cocoate, sold under the name Rewoderm LI-63 by the company Goldschmidt; and mixtures thereof.

The (poly)glycerol esters according to the invention are glycerol esters (or monoglyceryl esters) or polyglycerol esters (or polyglyceryl esters) such as diglyceryl (or diglycerol) esters.

According to one embodiment, the (poly)glycerol ester according to the invention results from the esterification of at least one saturated or unsaturated fatty acid and of a (poly)glycerol.

The term “(poly)glycerol” denotes glycerol or glyceryl polymers. When it is a polymer, the polyglycerol is generally a linear sequence of 1 to 22 and preferably of 1 to 12 glycerol units.

In the context of the present invention, the term “polyoxyalkylenated (poly)glycerol” corresponds to polyoxyalkylenated ethers of glycerol (or of polyglycerol) and preferably polyoxyethylenated (or polyethylene glycol) ethers.

The esters more particularly considered according to the present invention are esters resulting from the esterification of poly(glycerol) and of C₁₂-C₂₀, preferably C₁₂ to C₁₈, preferably C₁₂, carboxylic acid(s), for the fatty acid esters of polyglycerol (a), such as lauric, oleic, stearic, isostearic and myristic acids.

The esters more particularly considered according to the present invention are esters resulting from the esterification of poly(glycerol) and of C₆-C₁₈, preferably C₁₂ to C₁₈, preferably C₁₀-C₁₂, carboxylic acid(s), for the fatty acid esters of polyglycerol (b), such as capric, caprylic or lauric acids.

The carboxylic acid may be linear or branched, and saturated or unsaturated. Preferably, it is a linear monocarboxylic acid.

In general, they are derived from the esterification of at least one hydroxyl function of a poly(glycerol) with a C₁₂-C₂₀, preferably C₁₂ to C₁₈, and more particularly C₆ to C₁₈, in particular C₁₉ to C₁₂, carboxylic acid.

According to a particular embodiment, the esters that are suitable for use in the present invention may be derived from the esterification of a poly(glycerol) with one or more identical or different carboxylic acids. It may be a hydroxylated monoester, a hydroxylated diester, a hydroxylated triester, or a mixture thereof.

A preferred cosmetic composition according to the invention comprises an ester of (poly)glycerol chosen from the group constituted of glycerol and glycerol polymers.

In one preferred embodiment of the invention, the first fatty acid ester of polyglycerol a) is chosen from polyglyceryl monolaurate comprising from 4 to 6 glycerol units, polyglyceryl monooleate comprising from 4 to 6 glycerol units, polyglyceryl mono(iso)stearate comprising from 4 to 6 glycerol units, polyglyceryl monolaurate comprising from 4 to 6 glycerol units, polyglyceryl dioleate comprising from 4 to 6 glycerol units, polyglyceryl monomyristate comprising from 4 to 6 glycerol units, and mixtures thereof.

In one preferred embodiment of the invention, the second fatty acid ester of (poly)glycerol b) is chosen from (poly)glyceryl monolaurate comprising from 1 to 3 glycerol units, (poly)glyceryl monocaprate comprising from 1 to 3 glycerol units, (poly)glyceryl monocaprylate comprising from 1 to 3 glycerol units, (poly)glyceryl monostearate comprising from 1 to 3 glycerol units, and mixtures thereof.

In another preferred embodiment of the invention, the first fatty acid ester of polyglycerol a) has an HLB (hydrophilic lipophilic balance) value of 10 to 13, and/or the second fatty acid ester of (poly)glycerol b) has an HLB value of 8 to 10.

Advantageously, the composition according to the invention comprises a first fatty acid ester of polyglycerol a) which is a polyglyceryl monolaurate comprising 4 to 6 glycerol units, in particular PG-5 laurate, and the second fatty acid ester of (poly)glycerol b) is chosen from (poly)glyceryl monolaurate comprising from 1 to 3 glycerol units and (poly)glyceryl monocaprate comprising from 1 to 3 glycerol units, and is preferably chosen from PG-2 laurate and PG-2 caprate.

Preferably, the fatty acid ester of (poly)glycerol (a) is chosen from a mixture of fatty acid esters of (poly)glycerol, in particular formed from 3 to 6 glycerol units, preferably formed from 5 or 6 glycerol units, and in which the mixture preferably comprises at least 30% or more of fatty acid esters of (poly)glycerol comprising 5 to 6 glycerol units.

Preferably, the starting material of fatty acid esters of (poly)glycerol (a) present in the composition of the invention comprises fatty acid esters of polyglycerols containing 70% or more of polyglycerols of which the degree of depolymerization is 4 or more, fatty acid esters of polyglycerols containing at most 30% of polymerization of polyglycerols of which is 5.

Esters chosen from monoglyceryl and/or diglyceryl caprylate, monoglyceryl and/or diglyceryl heptanoate, monoglyceryl and/or diglyceryl caprylate, propylene glycol caprylate and propylene glycol heptanoate, and mixtures thereof, are most particularly suitable for use in the invention.

It is more particularly monoglyceryl caprylate (also known as glyceryl caprylate) and mixtures thereof.

In a particularly advantageous manner, the composition according to the invention comprises:
as first fatty acid ester of polyglycerol, polyglyceryl-5 laurate, and/or
as second fatty acid ester of (poly)glycerol, polyglyceryl-2 laurate or polyglyceryl-2 caprate.

Polyglyceryl-5 laurate or PG-5 laurate is available under the trade name Laurate Sunsoft A-121E® by the company Taiyo Kagaku.

Polyglyceryl-2 laurate or PG-2 laurate is available under the trade name Sunsoft Q-12D-C® by the company Taiyo Kagaku.

Polyglyceryl-2 caprate or PG-2 caprate is available under the trade name Sunsoft Q-10D-C® by the company Taiyo Kagaku.

In the compositions according to the invention, the total amount of fatty acid ester(s) of (poly)glycerol (a) may be from 0.5% to 20% by weight, preferably from 1% to 10% by weight and more preferably from 2% to 9% by weight, relative to the total weight of the composition.

In the compositions according to the invention, the total amount of fatty acid ester(s) of (poly)glycerol (b) may be from 0.1% to 20% by weight, preferably from 1% to 10% by weight and more preferably from 1.5% to 7% by weight, relative to the total weight of the composition.

In the compositions according to the invention, the total amount of the fatty acid esters of (poly)glycerol (a) and (b) present ranges from 0.5% to 40% by weight, preferably from 2% to 20% by weight and more preferably from 3.5% to 16% by weight, relative to the total weight of the composition.

More particularly, in the compositions according to the invention, the [total amount of fatty acid ester(s) of polyglycerol (a)] to [total amount of fatty acid ester(s) of (poly)glycerol (b)] weight ratio ranges from 0.2 to 10, in particular from 0.5 to 5, preferably from 1 to 2.

Polyol (c)

The composition according to the present invention comprises at least one polyol.

The total amount of polyol(s) present in the composition according to the invention is greater than or equal to 15% by weight relative to the total weight of the composition.

According to the invention, the term “polyol” is intended to mean a hydrocarbon-based chain comprising at least two carbon atoms, preferably from 2 to 50 carbon atoms, preferably from 4 to 20 carbon atoms, preferably containing from 2 to 10 carbon atoms and preferentially containing from 2 to 6 carbon atoms, and bearing at least two hydroxyl groups. The polyols used in the present invention may have a weight-average molecular weight of less than or equal to 1000 and preferably between 90 and 500.

The polyol may be a natural or synthetic polyol. The polyol may have a linear, branched or cyclic molecular structure.

The polyol may be chosen from glycerol and derivatives thereof, and glycols and derivatives thereof. The polyol may be chosen from the group constituted of glycerol, diglycerol, polyglycerol, diethylene glycol, propylene glycol, dipropylene glycol, butylene glycol, pentylene glycol, hexylene glycol, 1,3-propanediol, 1,5-pentanediol, 1,2-octanediols, polyethylene glycols, in particular containing from 5 to 50 ethylene oxide groups, and sugars such as sorbitol, and a mixture thereof.

More particularly, the polyol may be chosen from the group constituted of dipropylene glycol and butylene glycol, and a mixture thereof.

Said polyol(s) may be present in a content ranging from 15% to 60% by weight, preferably ranging from 20% to 40% by weight and preferentially ranging from 20% to 30% by weight, relative to the total weight of the composition.

Oil (d)

The cosmetic composition according to the present invention comprises at least one oil (d). According to the present invention, the term “oil” denotes a fatty compound or substance that is in the form of a liquid at ambient temperature (25° C.) and at atmospheric pressure (760 mmHg). As oils, those generally used in cosmetics may be used alone or in combinations thereof. These oils may be volatile or non-volatile, preferably non-volatile.

The oil may be a non-polar oil such as a hydrocarbon-based oil, a silicone oil or the like; a polar oil such as a plant or animal oil and an ester oil or an ether oil; or a mixture thereof.

It is preferable for the oil (d) to be chosen from the group constituted of oils of plant origin, animal oils, synthetic oils, silicone oils and hydrocarbon-based oils.

As examples of plant oils, mention may be made, for example, of linseed oil, camellia oil, macadamia oil, corn oil, castor oil, olive oil, avocado oil, sasanqua oil, safflower oil, jojoba oil, sunflower oil, almond oil, rapeseed oil, sesame oil, soybean oil, groundnut oil, argan oil and apricot kernel oil, and mixtures thereof.

As examples of animal oils, mention may be made, for example, of squalene and squalane.

As examples of synthetic oils, mention may be made of alkanes such as isododecane and isohexadecane, fatty esters, fatty ethers and artificial C₆-C₂₂ acid triglycerides.

The fatty esters are preferably liquid esters of linear or branched, saturated or unsaturated C₁-C₂₆ aliphatic monoacids or polyacids and of linear or branched, saturated or unsaturated C₁-C₂₆ aliphatic monoalcohols or polyalcohols, the total number of carbon atoms in the fatty esters being greater than or equal to 10.

Preferably, for the monoalcohol esters, at least one from among the alcohol and the acid is branched.

Among the monoesters of monoacids and of monoalcohols, mention may be made of ethyl palmitate, ethylhexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols and esters of monocarboxylic, dicarboxylic or tricarboxylic acids and of non-saccharide C₄-C₂₆ dihydroxy, trihydroxy, tetrahydroxy or pentahydroxy alcohols may also be used.

Mention may in particular be made of: diethyl sebacate; isopropyllauryl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

Fatty esters that may be used include sugar esters and diesters of C₆-C₃₀ and preferably C₁₂-C₂₂ fatty acids. It is recalled that the term “sugar” denotes hydrocarbon-based compounds comprising oxygen containing several alcohol functions, with or without aldehyde or ketone functions, and which comprise at least 4 carbon atoms. These sugars may be monosaccharides, oligosaccharides or polysaccharides.

Examples of suitable sugars that may be mentioned include saccharose (or sucrose), glucose, galactose, ribose, fucose, maltose, fructose, mannose, arabinose, xylose and lactose, and derivatives thereof, in particular alkyl derivatives such as methyl derivatives, for example methylglucose.

The sugar esters of fatty acids may be chosen in particular from the group comprising the esters or mixtures of esters of sugars described previously and of linear or branched, saturated or unsaturated C₆-C₃₀ and preferably C₁₂-C₂₂ fatty acids. If they are unsaturated, these compounds may contain from one to three conjugated or unconjugated double bonds.

The esters according to this variant can also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, in particular, oleopalmitate, oleostearate and palmitostearate mixed esters, and also pentaerythrityl tetraethyl hexanoate.

More particularly, use is made of monoesters and diesters and in particular sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As preferred examples of fatty esters, mention may be made, for example, of diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethyl hexanoate, 2-ethylhexyl octanoate, 2-ethylhexyl caprylate/caprate, methyl palmitate, ethyl palmitate, isopropyl palmitate, ethylhexyl palmitate, isohexyl laurate, hexyl laurate, isocetyl stearate, isopropyl isostearate, isopropyl myristate, isodecyl oleate, glyceryl tris(2-ethyl hexanoate), pentaerythrityl tetrakis(2-ethylhexanoate), 2-ethylhexyl succinate and diethyl sebacate, and mixtures thereof.

As examples of artificial triglycerides, mention may be made, for example, of glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made, for example, of linear organopolysiloxanes such as dimethylpolysiloxane, methylphenylpolysiloxane, methylhydrogenopolysiloxane and the like; cyclic organopolysiloxanes such as octamethylcyclotetrasiloxane, decamethylcyclopentasiloxane, dodecamethylcyclohexasiloxane and the like; and mixtures thereof.

Preferably, the silicone oil is chosen from liquid polydialkylsiloxanes, in particular liquid polydimethylsiloxanes (PDMS) and liquid polyorganosiloxanes comprising at least one aryl group.

These silicone oils may also be organomodified. The organomodified silicones that may be used according to the present invention are silicone oils as defined above comprising in their structure one or more organofunctional groups linked via a hydrocarbon-based group.

The organopolysiloxanes are defined in greater detail in Walter Noll's Chemistry and Technology of Silicones (1968), Academic Press. They may be volatile or non-volatile.

Volatile or non-volatile silicone oils, such as volatile or non-volatile polydimethylsiloxanes (PDMS) containing a linear or cyclic silicone chain, which are liquid or pasty at ambient temperature, in particular cyclopolydimethylsiloxanes (cyclomethicones) such as cyclohexasiloxane; polydimethylsiloxanes containing alkyl, alkoxy or phenyl groups that are pendent or at the end of the silicone chain, said groups containing from 2 to 24 carbon atoms; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenyl ethyl trimethylsiloxysilicates and polymethylphenylsiloxanes, may be used.

The hydrocarbon-based oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, a liquid paraffin gel, polydecenes and hydrogenated polyisobutenes such as Parleam, and squalane.

As preferred examples of hydrocarbon-based oils, mention may be made, for example, of linear or branched hydrocarbons such as mineral oil (for example liquid paraffin), paraffin, petroleum jelly or petrolatum, naphthalenes and the like; hydrogenated polyisobutene, isoeicosane, and a decene/butene copolymer; and mixtures thereof.

It is also preferable for the oil (d) to be chosen from oils with a molecular weight of less than 600 g/mol.

Preferably, the oil (d) is chosen from fatty esters containing one or more C₁-C₁₂ hydrocarbon-based chains (for example isopropyl myristate, isopropyl palmitate, isononyl isononanoate and ethylhexyl palmitate), hydrocarbon-based oils (for example isododecane, isohexadecane and squalane), oils of branched and/or unsaturated C₁₂-C₃₀ fatty alcohol type such as octyldodecanol or oleyl alcohol, and fatty ethers such as dicaprylyl ether.

In a particularly preferred manner, the oil (d) is chosen from ethylhexyl palmitate and isopropyl myristate.

Ethylhexyl palmitate is available under the trade name 2-ethylhexyl palmitate (DUB PO)® from the company Stéarineries Dubois.

The amount of oil(s) (d) in the cosmetic composition according to the present invention may be in the range from 0.50% to 80% by weight, in particular from 1% to 50% by weight, preferably from 1% to 15% by weight and more preferably from 2% to 6% by weight, relative to the total weight of the composition.

In one particular embodiment, the fatty acid ester(s) of (poly)glycerol (b) and the oil(s) (d) are present in a composition according to the invention, as defined above, in a [total amount of fatty acid ester(s) of (poly)glycerol (b)/oil(s) (d)] weight ratio ranging from 0.50 to 10, preferably ranging from 1 to 5.

Water (e)

The cosmetic composition according to the present invention comprises water.

The amount of water (e) is not limited, and may range from 30% to 90% by weight, preferably from 35% to 80% by weight and more preferably from 40% to 70% by weight, relative to the total weight of the composition.

The composition according to the present invention may also comprise at least one additional surfactant other than the fatty acid ester(s) of (poly)glycerol (a) and (b) as defined above.

Additional Anionic Surfactant (f)

The composition according to the present invention may also comprise at least one additional anionic surfactant other than the fatty acid ester(s) of (poly)glycerol (a) and (b) as defined above.

The composition of the present invention may comprise at least one sulfonate salt (fatty acid amide) anionic surfactant.

Said anionic surfactants(s) is (are) at least one sulfonate salt (fatty acid amide) surfactant represented by formula (II) below:

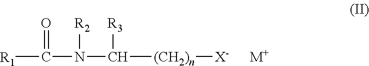

in which:

R₁ is a saturated or unsaturated, linear or branched alkyl chain having from 7 to 17 carbon atoms,

R₂ is H or a methyl group,

R₃ is H, COO⁻M⁺, MCH₂COO⁻ or COOH,

n is 0 to 2,

X represents COO or SO₃⁻ and

M represents independently H, sodium, potassium or sorbitan,

and mixtures thereof.

Such anionic surfactant agents are those described in EP 2 335 681.

Preferably, the anionic surfactants (f) are chosen from surfactants of the type taurate, glutamate, alanine or alanate, sarcosinate, aspartate, and mixtures thereof.

Preferably, the anionic surfactants are surfactants of the type taurate, glutamate, sarcosinate and/or mixtures thereof.

Particularly preferably, the anionic surfactants (f) are taurate surfactants.

In particular, the taurate surfactants are according to general formula (III)

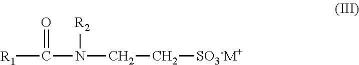

in which:

R₁ is preferably a saturated or unsaturated, linear or branched alkyl chain with from 7 to 17 carbon atoms, and more particularly from 9 to 13 carbon atoms,

R₂ is H or a methyl group,

and M is H, sodium or potassium.

The anionic surfactants of formula (III) can be chosen from potassium cocoyltaurate, potassium methylcocoyltaurate, sodium caproylmethyltaurate, sodium cocoyltaurate, sodium lauroyltaurate, sodium methylcocoyltaurate, sodium methyllauroyltaurate, sodium methylmyristoyltaurate, sodium methyloleoyltaurate, sodium methylpalmitoyltaurate, and sodium methylstearoyltaurate, and mixtures thereof.

More particularly, the anionic surfactant(s) of formula (III) are chosen from potassium cocoyltaurate, potassium methylcocoyltaurate, sodium cocoyltaurate, sodium lauroyltaurate, sodium methylcocoyltaurate and sodium methyllauroyltaurate, and mixtures thereof.

The compositions of the present invention may also comprise mixtures of anionic surfactants of amino acid type, such as the mixture of anionic surfactants of glutamate and taurate type, a mixture of taurates, or a mixture of surfactants of glutamate and sarcosinate type.

The term “anionic surfactant of amino acid type” is intended to mean surfactants which are derived from taurate, glutamate, alanine or alaninate, sarcosinate and aspartate.

According to one embodiment of the invention, at least one anionic surfactant is chosen from the group constituted of an isethionate, a taurate, a sarcosinate, a sulfosuccinate, a sulfoacetate, a glycinate, a glutamate and a carboxylate, in which at least one anionic surfactant has a C₈ to C₂₀ alkyl chain, and a solubilizing countercation chosen from sodium, potassium and ammonium.

According to one embodiment of the invention, at least one anionic surfactant agent is a taurate, said taurate having a C₈ to C₂₀ alkyl chain, and a solubilizing countercation chosen from sodium, potassium and ammonium.

According to one embodiment of the invention, at least one anionic surfactant agent is chosen from the group constituted of sodium laurylmethylisethionate, sodium methyloleoyltaurate, sodium N-myristoyl-N-methyltaurate, sodium (coconut fatty acid)methyltaurate and sodium laurylmethyltaurate.

According to one particularly preferred embodiment of the invention, the anionic surfactant is an anionic surfactant of formula (II) in which R₂ is a methyl, R₁ is a saturated linear alkyl chain having 17 carbon atoms, and M is sodium, i.e. sodium N-methylstearoyltaurate. Sodium N-methylstearoyltaurate is available under the trade name Nikkol SMT® by the company Nikko.

The total amount of the anionic surfactant(s) of formula (II), preferably of formula (III), may be from 0.01% to 2% by weight, in particular from 0.05% to 1% by weight, preferably from 0.08% to 0.5% by weight, relative to the total weight of the composition according to the invention.

Thickener

The cosmetic composition according to the present invention may also comprise at least one thickener.

The thickener may be chosen from organic and inorganic thickeners.

The thickener is preferably chosen from associative thickeners and polysaccharides such as starch and xanthan gum.

In the present context, the term “associative thickener” denotes an amphiphilic thickener comprising both hydrophilic and hydrophobic units, for example comprising at least one C₈-C₃₀ fatty chain and at least one hydrophilic unit.

The viscosity of the cosmetic composition according to the present invention is not particularly limited. The viscosity may be measured at 25° C. with viscometers or rheometers, preferably having cone-plate geometry. Preferably, the viscosity of the cosmetic composition according to the present invention may be, for example, from 1 to 2000 Pa·s and preferably from 1 to 1000 Pa·s at 25° C. and 1 s⁻¹.

The thickener may be present in an amount in the range from 0.001% to 10% by weight and preferably from 0.01% to 10% by weight, for example from 0.1% to 5% by weight, relative to the total weight of the composition.

Other Components

The cosmetic composition according to the present invention may also comprise an efficient amount of other components, previously known elsewhere in compositions, in particular cosmetic compositions, such as various adjuvants, anti-ageing agents, depigmenting agents, moisturizing agents, anti-greasy skin agents, sequestrants such as EDTA and etidronic acid, UV stabilizers, preserving agents (such as phenoxyethanol), vitamins or provitamins, for example, opacifiers, fragrances, plant extracts, cationic polymers, etc.

Preparation and Properties

The cosmetic composition according to the present invention may be prepared by mixing the essential and optional components above according to a conventional process. The conventional process comprises mixing with a high-pressure homogenizer (a high-energy process). As a variant, the cosmetic composition may be prepared via a low-energy process such as a phase inversion temperature (PIT) process, a phase inversion concentration (PIC), self-emulsification, and the like. Preferably, the cosmetic composition is prepared via a low-energy process.

In a particular embodiment, the weight ratio between the total amount of the fatty acid ester(s) of (poly)glycerol defined in (a) and (b) and the oil (d) in a composition according to the invention as defined above ranges from 0.50 to 10, preferably from 1 to 5.

The cosmetic composition according to the present invention is in the form of a nanoemulsion or microemulsion.

The term “microemulsion” may be defined in two ways, i.e. in a broad sense and in a narrower sense. Namely, in one case (“microemulsion in the narrow sense”), the term microemulsion denotes a thermodynamically stable isotropic single liquid phase containing a ternary system having three components comprising an oily component, an aqueous component and a surfactant, and, in the other case (“microemulsion in the broad sense”), among the thermodynamically unstable typical emulsion systems, the term microemulsion also comprises emulsions that have transparent or translucent appearances on account of their smaller particle sizes (Satoshi Tomomasa, et al., Oil Chemistry, vol. 37, No. 11 (1988), p. 48-53). In the present context, the term “microemulsion” denotes a “microemulsion in the narrow sense”, i.e. a thermodynamically stable isotropic single liquid phase.

The microemulsion denotes a state of a microemulsion of O/W (oil-in-water) type in which the oil is dissolved by micelles, a microemulsion of W/O (water-in-oil) type in which the water is dissolved by inverse micelles, or a bicontinuous microemulsion in which the number of associations of surfactant molecules tends to infinity such that the aqueous phase and the oily phase both have a continuous structure.

The microemulsion may have a dispersed phase with a number-average diameter of 100 nm or less, preferably 50 nm or less and more preferably 20 nm or less, measured by laser particle size analysis.

The term “nanoemulsion” presently denotes an emulsion characterized by a dispersed phase with a size of less than 350 nm, the dispersed phase being stabilized by a crown of the non-ionic surfactants (a) and (b) which may optionally form a liquid crystal phase of lamellar type, at the dispersed phase/continuous phase interface. In the absence of specific opacifiers, the transparency of nanoemulsions is due to the small size of the dispersed phase, this small size being able to be obtained by means of using mechanical energy and in particular a high-pressure homogenizer. In one particular embodiment of nanoemulsion according to the invention, said nanoemulsion is stabilized by, in addition to the non-ionic surfactants (a) and (b), the anionic surfactant(s) (f) which in particular allow repulsion between the drops of dispersed phase.

Nanoemulsions may be distinguished from microemulsions by their structure. Specifically, microemulsions are thermodynamically stable dispersions formed, for example, from swollen micelles of non-ionic surfactants (a) and (b) with the oil (d). Furthermore, microemulsions do not require substantial mechanical energy to be prepared.

The microemulsion may have a dispersed phase with a number-average diameter of 300 nm or less, preferably 200 nm or less and more preferably 100 nm or less, measured by laser particle size analysis.

The cosmetic composition according to the present invention may be in the form of an O/W nanoemulsion or microemulsion, a W/O nanoemulsion or microemulsion, or a bicontinuous emulsion. It is preferable for the cosmetic composition according to the present invention to be in the form of an O/W nanoemulsion or microemulsion.

It is preferable for the cosmetic composition according to the present invention to be in the form of an O/W emulsion.

The mean size of the droplets of the oily phase is measured concentrated by dynamic light scattering (DLS) with a Vasco particle size analyser.

These measurements are taken on the undiluted emulsion.

The number-average size (μm) of the droplets of oily phase of the composition of the invention is less than 300 nm, preferably from 10 nm to 150 nm and more preferably from 20 nm to 100 nm.

The cosmetic composition according to the present invention may have a transparent or slightly translucent appearance, preferably a transparent appearance.

The transparency may be measured by measuring the transmission factor with an absorption spectrometer in the visible region (for example, the transparency is measured with a Hach 2100Q portable turbidimeter at 25° C.). The portable turbidimeter uses the nephelometric principle for measuring turbidity. The nephelometric turbidity measurement depends on the detection of the light scattered by the particles in suspension in the liquid. The measuring unit is the NTU. A borosilicate glass round cuvette 60×25 cm with a screw cap is used. The amount of sample required is 15 ml. The measuring range is 0-1000 NTU. The samples are measured undiluted.

The cosmetic composition according to the present invention may preferably have a turbidity of between 1 and 200 NTU and preferably between 5 and 100 NTU.

Process and Use

A further subject of the invention is a process for the cosmetic treatment of keratin materials, comprising the application to the keratin materials, in particular the skin, of a composition according to the invention as described previously.

Said cosmetic treatment process is non-therapeutic.

In one embodiment, said composition according to the invention does not comprise hesperetin.

More particularly, a subject of the invention is also a cosmetic treatment process for caring for, making up and/or cleansing keratin materials, in particular the skin, comprising the application to said keratin materials, in particular the skin, of a composition according to the invention as described previously.

Said cosmetic treatment process for caring for, making up and/or cleansing the skin is non-therapeutic.

More particularly, a subject of the invention is also a non-therapeutic cosmetic process for preventing and/or treating the signs of skin ageing, comprising at least one step of topical application to the keratin materials, such as the skin, of a composition according to the invention as described previously.

The signs of skin ageing to be prevented and/or treated in the cosmetic process according to the invention may be chosen from wrinkles and fine lines, skin exhibiting impairment of its viscoelastic or biomechanical properties, skin exhibiting impairment in the cohesion of its tissues, thinned skin, and skin exhibiting impairment of its surface appearance.

More particularly, a further subject of the invention is a cosmetic process for preventing and/or treating the signs of skin ageing, comprising at least one step of topical application to the keratin materials, such as the skin, of a composition according to the invention as described previously, characterized in that it is intended for promoting keratinocyte renewal and for reducing or preventing signs chosen from thinning of the epidermis, surface wrinkles, impairments of the barrier function, the properties of stretchability, tonicity, firmness, suppleness and/or elasticity of the skin.

A subject of the invention is also the cosmetic use of a composition according to the invention as defined previously, for caring for, making up and/or cleansing keratin materials.

More particularly, a subject of the present invention is also the use of a composition according to the invention as defined above, for preventing and/or reducing the signs of skin ageing, in particular the signs on the skin chosen from wrinkled skin, skin exhibiting impairment of its viscoelastic or biomechanical properties, skin exhibiting impairment in the cohesion of its tissues, thinned skin, and skin exhibiting impairment of its surface appearance.

The use according to the invention is a non-therapeutic use and advantageously a cosmetic use; the term “cosmetic” means intended to improve the aesthetic appearance of keratin materials, such as the skin or the nails, and in particular to retard or reduce physiological modifications in the appearance, arising with age, of individuals in good health. These modifications may appear from the age of 30 or 35, but are generally more pronounced after the age of 40 (mature skin), and become accentuated at 50 and over.

The compositions according to the invention are effective for improving epidermal renewal and for preventing and/or treating the signs of skin ageing, in particular topically, and most particularly the signs on the skin related to wrinkled skin, skin exhibiting impairment of its viscoelastic or biomechanical properties, skin exhibiting impairment in the cohesion of its tissues, thinned skin and/or skin exhibiting impairment of its surface appearance.

In fact, it has now been found that compositions according to the invention in the form of a nanoemulsion or microemulsion, comprising at least two different fatty acid esters of (poly)glycerol, at least 15% of polyol(s), at least one oil, water, and optionally at least one anionic surfactant of formula (II), are capable of stimulating filaggrin expression, and/or of preventing a decrease in the thickness of the epidermis.

Such compositions thus prove to be particularly advantageous for preventing and/or treating the signs of skin ageing, as explained above, in particular filaggrin expression being involved in the formation of the stratum corneum.

The use of the compositions according to the invention may make it possible more particularly to maintain and/or restore the biomechanical properties of the skin.

The term “biomechanical properties of the skin” is intended to mean herein the stretchability, tonicity, firmness, suppleness and/or elasticity properties of the skin.

The term “signs of skin ageing” is intended to mean herein any modification of the outer appearance of the skin due to ageing, whether it is chronobiological and/or extrinsic ageing, in particular photoinduced or hormonal ageing; among these signs, it is possible to distinguish:
wrinkled skin, which is reflected in particular by the appearance of wrinkles and/or fine lines;
skin exhibiting impairment of its viscoelastic or biomechanical properties, or skin exhibiting a lack of elasticity and/or of stretchability and/or of firmness and/or of suppleness and/or of tonicity, which is reflected in particular by wizened, flaccid, slack or saggy skin;
skin exhibiting impairment of the cohesion of its tissues;
thinned skin; and
skin exhibiting impairment of its surface appearance, which is in particular reflected by impairment of the grain of the skin, for example roughness.

The invention relates to the non-therapeutic use of a composition according to the invention for preventing and/or reducing the signs of skin ageing, in particular the signs on the skin chosen from wrinkled skin, skin exhibiting impairment of its viscoelastic or biomechanical properties, skin exhibiting impairment in the cohesion of its tissues, thinned skin, and skin exhibiting impairment of its surface appearance.

According to the invention, the term “keratin materials” is intended to mean the skin, of the body, face and/or area around the eyes, the lips, the nails, the mucous membranes, or any other area of bodily skin. More particularly, the keratin material according to the invention is the skin.

The term “skin” is intended to mean all of the skin of the body, and preferably the skin of the face, neckline, neck, arms and forearms, or even more preferably still the skin of the face, in particular of the forehead, nose, cheeks, chin and area around the eyes.

As specified hereinbelow, hesperetin is advantageously present in the compositions in accordance with the invention in a dissolved form.

By way of example, the composition according to the invention may be intended to be administered topically, i.e. by application at the surface of the keratin material under consideration, such as the skin under consideration, optionally by application of a transdermal patch containing it.

The cosmetic composition according to the present invention may be used for a non-therapeutic process, such as a cosmetic process, for treating the skin, the mucous membranes, the nails or the eyelids, by application to the skin, the mucous membranes, the nails or the eyelids.

The present invention also relates to a use of the cosmetic composition according to the present invention, in its native form or in care products and/or washing products and/or makeup products and/or makeup-removing products for bodily and/or facial skin and/or the mucous membranes and/or the nails and/or the eyelids.

The care product may be a lotion, a cream, a hair tonic, a hair conditioner, a sunscreen, and the like. The cleansing product may be a facial cleanser, a hand cleanser, and the like. The makeup product may be a foundation, a mascara, a lipstick, a lip gloss, a face powder, an eyeshadow, a nail varnish, and the like. The makeup-removing product may be a makeup-cleansing product, and the like.

A composition according to the invention is advantageously an anti-ageing composition, in particular a care composition for treating and/or combating, in particular cosmetically, the external signs of skin ageing.

The composition is more particularly a composition for caring for mature skin.

The expressions “between . . . and . . . ” and “ranging from . . . to . . . ” or “at least . . . or “at the least . . . ” should be understood as being limits inclusive, unless otherwise specified.

The present invention is described in greater detail by means of examples, which should not, however, be considered as limiting the scope of the present invention.

The compounds are indicated as their chemical name or their INCI name.

The amounts of the ingredients are expressed as weight percentages.

### EXAMPLE 1

### Cosmetic Composition According to the Invention

A facial care lotion having the following composition was prepared:

| | | % by weight |
|---|---|---|
| | | relative to the |
| | | total weight of |
| Phases | Ingredients | the composition |
| A | Polyglyceryl-2 laurate or PG-2 laurate | 2 |
| | (Sunsoft Q-12D-C ® from Taiyo Kagaku) | |
| | Polyglyceryl-5 laurate or PG-5 laurate | 3 |
| | (Sunsoft A-121E ® from Taiyo Kagaku) | |
| | 2-Ethylhexyl palmitate (2-Ethylhexyl | 5 |
| | palmitate (Cegesoft C24 ® from | |
| | BASF) | |
| | Sodium N-methylstearoyltaurate or sodium | 0.1 |
| | methyl stearoyl taurate (Nikkol SMT ® from | |
| | Nikko) | |
| B | water | qs 100 |
| C | Butylene glycol | 20 |
| | (1,2-Propylene glycol Care ® from BASF) | |
| D | Phenoxyethanol | 0.5 |
| | 1,2-Octanediol (Minacare Octiol ® from | 0.5 |
| | Minasolve) | |

Preparation Method:

(1) ethylhexyl palmitate, sodium N-methylstearoyltaurate, polyglyceryl-5 laurate and polyglyceryl-2 laurate were mixed to form an oily phase A;

(2) the oily phase A was heated to about 70° C.;

(3) water B was added to the oily phase A with stirring to obtain an oil-in-water microemulsion;

(4) the polyol (butylene glycol) was mixed, and then solution C was added to the microemulsion;

(5) phase D (phenoxyethanol and 1,2-octanediol) was added.

The composition is single-phase and clear, and remains stable and homogeneous after storage for 3 months at 40° C.

This composition may be applied regularly to facial skin in order to attenuate the signs of skin ageing.

### EXAMPLE 2

### Cosmetic Composition According to the Invention

A facial care lotion having the following composition was prepared:

| | | % by weight |
|---|---|---|
| | | relative to the |
| | | total weight of |
| Phases | Ingredients | the composition |
| A | Polyglyceryl-2 caprate or PG-2 caprate | 6 |
| | (Sunsoft Q-10D-C ® from Taiyo Kagaku) | |
| | Polyglyceryl-5 laurate or PG-5 laurate | 8 |
| | (Sunsoft A-121E ® from Taiyo Kagaku) | |
| | Isopropyl myristate (Isopropylmyristate ® | 4 |
| | from BASF) | |
| B | water | qs 100 |
| C | Dipropylene glycol | 25 |
| | (Dipropylene glycol LO+ ® from Dow | |
| | Chemical) | |
| D | 1,2-Octanediol (Minacare Octiol ® from | 0.5 |
| | Minasolve) | |
| | Phenoxyethanol | 0.5 |
| | ethanol | 10 |

Preparation Method

(1) isopropyl myristate, polyglyceryl-5 laurate and polyglyceryl-2 caprate were mixed to form an oily phase A;

(2) the oily phase A was heated to about 70° C.;

(3) water B was added to the oily phase A with stirring to obtain an oil-in-water microemulsion;

(4) the polyol (dipropylene glycol) was mixed, and then solution C was added to the microemulsion;

(5) phase D (phenoxyethanol, ethanol and 1,2-octanediol) was added.

The composition is single-phase and clear, and remains stable and homogeneous after storage for 3 months at 40° C.

This composition may be applied regularly to facial skin in order to attenuate the signs of skin ageing and in particular to prevent or treat thinned skin.

### EXAMPLE 3

### Evaluation of the Beneficial Effects of a Composition According to the Invention on the Signs of Skin Ageing, by Evaluating Filaggrin Expression and the Thickness of the Epidermis

The beneficial effects of a composition according to the invention on the signs of skin ageing on a mature skin were evaluated using the model of Voorhees et al. (British Journal of dermatology, 1993, 129, 389-392).

Material and Method:

29 subjects (10 men/19 women) exhibiting photo-aged skin on the forearms (score>5 on the McKenzie scale) were treated with topical application by patch of various compositions for 12 hours, with:
composition 2 according to the invention in the form of a microemulsion (composition of Example 2)
comparative example 3, outside the invention, which is a composition in conventional emulsion form comprising 0.1% of retinol by weight
   comparative example 4, outside the invention, which is a composition identical to that of comparative example 3, conventional emulsion, but without the retinol active agent
an empty patch without composition.

Applications of 30 microlitres of formulas were carried out under patches for 12 consecutive days, the patches having been renewed every 4 days. Upon final removal of the patches (day 13), skin biopsies 2 mm in diameter were carried out on the application sites.

The skin samples were fixed in a phosphate buffer containing 10% formaldehyde, embedded in paraffin and treated for histological analysis. Part of the sections were cut at 5 micrometres, mounted on slides and stained with haematoxylineosin, according to standard procedures.

The slides are photographed digitally, and the thickness of the epidermis is automatically measured by dedicated software at 15 measurements per field, each slide having two fields.

The other paraffin sections that were mounted on slides are deparaffinized in xylene, rehydrated and prepared for immunolabelling by immunoperoxidase according to standard procedures.

After incubation with anti-filaggrin primary antibodies, the slides were washed and incubated with the biotinylated secondary antibody. The slides were then washed and treated with avidin-biotin-peroxidase for 30 minutes at ambient temperature. The slides were then revealed with 0.05% 3,39-diaminobenzidine and 0.03% H₂O₂.

The slides are photographed digitally, and the thickness of the epidermis labelled with the antibody is automatically measured by dedicated software at 15 measurements per field, each slide having two fields.

The results hereinafter are expressed by calculating the mean and the standard deviation on all of the measurements carried out. The statistical comparisons between treatments were carried out using repeated-measurement mixed models (ANOVA). A comparison is considered to be significant for a p<0.05.

The compositions of comparative examples 3 and 4 are compositions in emulsion form, but are not microemulsions or nanoemulsions as defined according to the invention, comprising respectively 0.1% of retinol which is a well-known anti-ageing active agent, or 0% of retinol.

The numerical values for the amounts of the components described in the table hereinafter are all based on weight percentages of starting materials relative to the total weight of the composition.

| | | Comparative | |
|---|---|---|---|
| | | example | |
| | | 3 (outside | Comparative |
| | | the invention) | example |
| | | comprising 0.1% | 4 (outside the |
| | Ingredients | of retinol | invention) |
| D | Sodium hydroxide | 0.2 | 0.2 |
| B | Preserving agent | 1 | 1 |
| A | CAPRYLIC/CAPRIC | 8 | 8 |
| | TRIGLYCERIDE (ERIDES | | |
| | C8C10 70/30 (DUB MCT | | |
| | 7030) ® from | | |
| | STEARINERIE DUBOIS) | | |
| A | APRICOT KERNEL OIL | 6 | 6 |
| | (APRICOT KERNEL OIL | | |
| | REFINED ® from GUSTAV | | |
| | HEESS) | | |
| A | CETYL ALCOHOL | 0.5 | 0.5 |
| | (LANETTE 16 ® from | | |
| | BASF) | | |
| A | MYRISTYL MYRISTATE | 2 | 2 |
| | (TEGOSOFT MM ® from | | |
| | EVONIK GOLDSCHMIDT) | | |
| D | ACRYLATES/C10-30 | 0.5 | 0.5 |
| | ALKYL ACRYLATE | | |
| | CROSSPOLYMER | | |
| | (CARBOPOL ULTREZ | | |
| | 20 ® POLYMER from | | |
| | LUBRIZOL) | | |
| B | WATER | qs | qs |
| A | STEARIC ACID (STEARIC | 1.5 | 1.5 |
| | ACID 1850 ® from | | |
| | SOUTHERN ACIDS) | | |
| A | SODIUM STEAROYL | 1 | 1 |
| | GLUTAMATE (AMISOFT | | |
| | HS 11 PF ® from | | |
| | AJINOMOTO) | | |
| E | RETINOL (RETINOL 10 | 0.1 | |
| | S ® from the company | | |
| | BASF) | | |

Preparation Method:

The preparation must be carried out in an oxygen-free atmosphere (under nitrogen) in order to avoid degradation of the retinol. Heat phase A and phase B at 80° C. Prepare the pre-gel of phase D by dusting the polymer onto the water while stirring using a deflocculator (Rayneri) and neutralize with sodium hydroxide. While stirring using a deflocculator (Rayneri), add phase D to phase B when the gel is uniform (temperature 38° C.), add phase A while stirring using a deflocculator (Rayneri) to phase B+D at a speed of 1000 rpm for 3 min, then at a temperature<30° C., add the retinol (E).

These formulas of comparative examples 3 and 4 are stable for three months at ambient temperature or stored at 40° C.

Results:

Histological Evaluation of Filaggrin Expression

Filaggrin expression (labelled epidermal thickness, μm)

| Application of | Mean | Standard deviation |
|---|---|---|
| Composition 2 according | 27.53 | 17.93 |
| to the invention | | |
| Comparative example | 25.50 | 9.84 |
| 3 (outside the invention) | | |
| comprising 0.1% | | |
| of retinol | | |
| Comparative example | 16.16 | 10.99 |
| 4 (outside the invention) | | |
| not comprising | | |
| retinol | | |
| empty patch without | 12.24 | 7.77 |
| composition | | |

The parameter measured is filaggrin expression by measuring the thickness of epidermis labelled with the antibody.

It was noted that the filaggrin expression is significantly higher for the skins on which the patch with composition 2 according to the invention of microemulsion type was applied, compared with an application with a patch comprising a composition outside the invention (comparative example 4) of conventional emulsion type not comprising retinol (that is to say not comprising anti-ageing active agents), and also compared with the application with a patch not comprising composition (p<0.05).

It was also noted, unexpectedly, that the filaggrin expression for the skins on which the patch with composition 2 according to the invention of microemulsion type was applied is not significantly different compared with an application with a patch comprising a composition outside the invention (comparative example 3) comprising 0.1% of retinol (which is a known anti-ageing active agent) (p=0.8), that is to say composition 2 according to the invention in microemulsion form allows filaggrin expression that is as good as a composition of conventional emulsion type comprising an anti-ageing active agent (retinol).

Histological Evaluation of the Thickness of the Epidermis

Epidermal thickness (μm)

| Application of: | Mean | Standard deviation |
|---|---|---|
| Composition 2 according | 79.36 | 21.91 |
| to the invention | | |
| Comparative example 3 | 84.00 | 24.76 |
| (outside the invention) | | |
| comprising 0.1% of retinol | | |
| Comparative example 4 | 66.86 | 32.13 |
| (outside the invention) | | |
| not comprising retinol | | |
| empty patch without | 66.00 | 23.79 |
| composition | | |

The parameter measured is the epidermal thickness.

It was noted that the thickness of the epidermis is significantly higher after the application of the patch with composition 2 according to the invention of microemulsion type, compared with the application with a patch not comprising composition (p<0.05).

It was also noted, unexpectedly, that the thickness of the epidermis for the skins on which the patch with composition 2 according to the invention of microemulsion type was applied is not significantly different compared with an application with a patch comprising a composition outside the invention (comparative example 3) comprising 0.1% of retinol (which is a known anti-ageing active agent) (p=0.25), that is to say composition 2 according to the invention in microemulsion form makes it possible to obtain an epidermal thickness similar to a composition of conventional emulsion type comprising an anti-ageing active agent (retinol).

Thus, unexpectedly, a composition according to the invention, in microemulsion or nanoemulsion form, and which does not comprise anti-ageing active agents such as retinol, is significantly as effective as a composition of conventional emulsion type comprising 0.1% of retinol, for preventing or reducing the decrease in the thickness of the epidermis, and thus as effective for preventing and treating the signs of skin ageing.

### EXAMPLE 4

### Evaluation of the Beneficial Effects of a Composition According to the Invention on the Signs of Skin Ageing such as Wrinkles and Firmness or Tonicity of the Skin

The beneficial effects of a composition according to the invention on the signs of skin ageing on mature or very mature skin were evaluated by clinical evaluation and instrumental measurements.

Material and Method:

80 women aged 40 to 65, exhibiting clinical signs of age, were treated every day with the products under investigation, on the face for 6 months, with:
compostion 1 according to the invention in the form of a microemulsion (composition of Example 1).

The 80 subjects return to the investigation centre after 7, 14, 28, 56, 84 and 168 days of treatment in order for clinical and instrumental evaluations to be carried out.

One of the signs of skin ageing evaluated is in particular the crows feet wrinkles, which are wrinkles located at the outer corners of the eyes.

Results

At each evaluation visit, a trained expert scores the severity of the wrinkles of the subjects having been treated with composition 1, according to a photographic scale graded from 1 to 6.

The results are expressed by calculating the mean of the scores of all the subjects evaluated. The statistical comparisons between treatments were carried out using repeated-measurement mixed models (ANOVA). A comparison is considered to be significant for a p<0.05.

Crows Feet Wrinkles: Variation of the Clinical Score Compared with the First Day of Study

| Days | mean | p.value |
|---|---|---|
| Day 07 | −0.040 | 0.713 |
| Day 14 | −0.355 | 0.001 |
| Day 28 | −0.355 | 0.001 |
| Day 56 | −0.461 | 0.000 |
| Day 84 | −0.592 | 0.000 |
| Day 168 | −0.671 | 0.000 |

It was noted that when composition 1 according to the invention is applied to the facial skin, the improvement in the score of the crows feet wrinkles is significant compared to the initial state, as early as 14 days of treatment (p<0.001), this being up to the end of the treatment at 6 months.

Composition 1 according to the invention applied to the skin makes it possible to reduce the skin wrinkles, and in particular the crows feet wrinkles of the facial skin.

Instrumental measurements of facial skin tonicity were also carried out using an apparatus dedicated to viscoelastic measurement of the skin, the cutometer (Courage and Khazaka, Germany). The probe is placed in contact with the skin on the cheek of the subjects and the tonicity measurement is carried out instantaneously. The R5 tonicity values are then used for the data analysis.

The results are expressed by calculating the mean of the R5 values of all the subjects evaluated. The statistical comparisons between treatments were carried out using repeated-measurement mixed models (ANOVA). A comparison is considered to be significant for a p<0.05.

Tonicity: Variation of the R5 Parameter Compared with the First Day of Study

| Days | average | p.value |
|---|---|---|
| Day 07 | 0.024 | 0.094 |
| Day 14 | 0.023 | 0.118 |
| Day 28 | −0.008 | 0.564 |
| Day 56 | 0.072 | 0.000 |
| Day 84 | 0.084 | 0.000 |
| Day 168 | 0.090 | 0.000 |

It was noted that when composition 1 according to the invention is applied to the facial skin, the improvement in the tonicity value measured is significant compared to the initial state, as early as 56 days of treatment (p<0.001), this being up to the end of the treatment at 6 months.

Composition 1 according to the invention applied to the skin makes it possible to improve the viscoelastic properties of the skin, and in particular the tonicity of the skin, in particular of facial skin.

Firmness

Composition 1 according to the invention applied to the skin makes it possible to improve the firmness of the skin, and in particular its efficacy on the improvement of the firmness of the skin is significantly better compared to the composition of the exemple 4, which is a conventional emulsion (outside the invention) when it is applied to the skin.

1. A composition in the form of a nanoemulsion or microemulsion, comprising:
a) a first fatty acid ester of polyglycerol which is chosen from a fatty acid ester of polyglycerol formed from at least one acid comprising an alkyl or alkenyl chain containing from 12 to 20 carbon atoms and from 3 to 6 glycerol units;
b) a second fatty acid ester of (poly)glycerol which is chosen from a fatty acid ester of (poly)glycerol formed from at least one acid comprising an alkyl or alkenyl chain containing from 6 to 18 carbon atoms and from 1 to 3 glycerol units;
c) at least one polyol in a total content of greater than or equal to 15% by weight relative to the total weight of the composition;
D) at least one oil;
e) water; and
f) at least one anionic surfactant of formula (II) 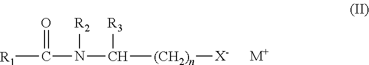
in which:
R₁ is a, saturated or unsaturated, linear or branched alkyl chain containing from 7 to 17 carbon atoms,
R₂ is H or a methyl,
R₃ is H, COO⁻M⁺, CH₂COO⁻M⁺ or COOH,
n is from 0 to 2,
X is COO⁻ or SO₃⁻ and
M represents independently H, sodium, potassium or sorbitan.

2. The composition according to claim 1, wherein the first fatty acid ester of polyglycerol a) is chosen from polyglyceryl monolaurate comprising from 4 to 6 glycerol units, polyglyceryl monooleate comprising from 4 to 6 glycerol units, polyglyceryl mono(iso)stearate comprising from 4 to 6 glycerol units, polyglyceryl monolaurate comprising from 4 to 6 glycerol units, polyglyceryl dioleate comprising from 4 to 6 glycerol units, polyglyceryl monomyristate comprising from 4 to 6 glycerol units, and mixtures thereof.

3. The composition according to claim 1, wherein the second fatty acid ester of (poly)glycerol b) is chosen from (poly)glyceryl monolaurate comprising from 1 to 3 glycerol units, (poly)glyceryl monocaprate comprising from 1 to 3 glycerol units, (poly)glyceryl monocaprylate comprising from 1 to 3 glycerol units, (poly)glyceryl monostearate comprising from 1 to 3 glycerol units, and mixtures thereof.

4. The composition according to claim 1, wherein the first fatty acid ester of polyglycerol a) has an HLB value of 10 to 13, and/or in that the second fatty acid ester of (poly)glycerol b) has an HLB value of 8 to 10.

5. Composition according to claim 1, wherein the first fatty acid ester of polyglycerol a) is a polyglyceryl monolaurate comprising 4 to 6 glycerol units and the second fatty acid ester of (poly)glycerol b) is chosen from (poly)glyceryl monolaurate comprising from 1 to 3 glycerol units and (poly)glyceryl monocaprate comprising from 1 to 3 glycerol units.

6. The composition according to claim 1, wherein it comprises at least one anionic surfactant of formula (III): 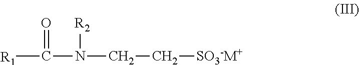
in which:
R₁ is preferably a saturated or unsaturated, linear or branched alkyl chain with from 7 to 17 carbon atoms, and more preferably from 9 to 13 carbon atoms,
R₂ is H or methyl,
and M is H, sodium or potassium, preferably the anionic surfactant of formula (III) is sodium N-methylstearoyltaurate.

7. The composition according to claim 1, in which the total amount of the fatty acid esters of (poly)glycerol (a) and (b) present ranges from 0.5% to 40% by weight relative to the total weight of the composition.

8. The composition according to claim 1, wherein said polyol (c) is chosen from glycerol, diglycerol, polyglycerol, propylene glycol, butylene glycol, pentylene glycol, hexylene glycol, dipropylene glycol, diethylene glycol, 1,3-propanediol, 1,5-pentanediol, polyethylene glycols, and sugars and a mixture thereof.

9. The composition according to claim 1, wherein said polyol(s) are present in a content ranging from 15% to 60% by weight relative to the total weight of the composition.

10. The composition according to claim 1, wherein said oil(s) (d) is (are) chosen from the group constituted of oils of plant origin, mineral oils, synthetic oils, silicone oils and hydrocarbon-based oils.

11. The composition according to claim 1, wherein the [total amount of fatty acid ester(s) of polyglycerol (a)] to [total amount of fatty acid ester(s) of (poly)glycerol (b)] weight ratio ranges from 0.2 to 10.

12. The composition according to claim 1, in which the oil(s) (d) is (are) present in a content of from 0.50% to 50% by weight relative to the total weight of the composition.

13. The composition according to claim 1, in which the [total amount of the fatty acid esters of (poly)glycerol (a) and (b)] to [oil(s) (d)] weight ratio ranges from 0.50 to 10.

14. The composition according to claim 1, wherein the anionic surfactant(s) of formula (II), preferably of formula (III), is (are) present in a total content of from 0.01% 2% by weight relative to the total weight of the composition.

15. The composition according to claim 1, wherein it is in the form of an oil-in-water (O/W) emulsion, and the oil (d) is in the form of droplets with a number-average particle size of 300 nm or less.

16. The composition according to claim 1, which is a cosmetic composition.

17. A cosmetic process for preventing and/or reducing the signs of skin ageing which comprises applying to the skin a composition as defined according to claim 1.

18. A cosmetic process for treating keratin materials, comprising the application to the keratin materials of a composition according to claim 1.

19. The cosmetic process according to claim 18, for reducing and/or preventing the signs of ageing of keratin materials wherein the composition is applied to mature and/or wrinkled skin.

20. The cosmetic process according to claim 19, which is intended for promoting the renewal of the keratinocytes and for reducing or preventing signs chosen from thinning of the epidermis, surface wrinkles, impairments of the barrier function, the properties of stretchability, tonicity, firmness, suppleness and/or elasticity of the skin.
